SEQUENCE LISTING (1 sequence)

SEQ ID NO: 1 - protein, 37 aa, Artificial sequence
  synthetic
EVVPPQVLSEPNEEAGAALSPLPEVPPWTGEVSPAQR

(19) United States
(12) Patent Application Publication
(10) Pub. No.: US 2022/0296680 A1
(43) Pub. Date: Sep. 22, 2022

(54) TREATMENT AND PREVENTION OF CARDIORENAL DAMAGE

(71) Applicants: Oslo Universitetssykehus HF, Oslo (NO); Universitetet i Oslo, Oslo (NO)

(72) Inventors: Raffaele Altara, Oslo (NO); Alessandro Cataliotti, Høvik (NO)

(21) Appl. No.: 17/700,836

(22) Filed: Mar. 22, 2022

Related U.S. Application Data
(60) Provisional application No. 63/164,054, filed on Mar. 22, 2021.

(51) Int. Cl. A61K 38/18; A61K 31/41; A61P 9/00

(52) U.S. Cl. CPC A61K 38/1883; A61K 31/41; A61P 9/00

(57) ABSTRACT

The present disclosure provides compositions and methods for treatment and prevention of cardiorenal damage. In particular, provided herein are compositions comprising proANP₃₁₋₆₇ for use in treating and preventing cardiorenal damage in subjects with heart failure.

### CROSS REFERENCE TO RELATED APPLICATIONS

This application claims the benefit of U.S. Prov. Appl. 63/164,054, filed Mar. 22, 2021, the entire contents of which are incorporated herein by reference.

### REFERENCE TO AN ELECTRONIC SEQUENCE LISTING

The contents of the electronic sequence listing (File Name: 39379-202_ST25.txt; Size: 1,000 bytes; and Date of Creation: Mar. 22, 2022), submitted on Mar. 22, 2022, is herein incorporated by reference in its entirety.

### FIELD OF THE INVENTION

The present disclosure provides compositions and methods for treatment and prevention of cardiorenal damage. In particular, provided herein are compositions comprising proANP₃₁₋₆₇ for use in treating and preventing cardiorenal damage in subjects with heart failure.

### BACKGROUND OF THE INVENTION

Heart failure with preserved ejection fraction (HFpEF), also referred to as diastolic heart failure, causes almost one-half of the 5 million cases of heart failure in the United States. It is more common among older patients and women, and results from abnormalities of active ventricular relaxation and passive ventricular compliance, leading to a decline in stroke volume and cardiac output.

Symptoms include fatigue, weakness, dyspnea, orthopnea, paroxysmal nocturnal dyspnea, edema, S3 heart sound, displaced apical pulse, and jugular venous distension. Echocardiographic findings of normal ejection fraction with impaired diastolic function confirm the diagnosis. Measurement of natriuretic peptides is useful in the evaluation of patients with suspected heart failure with preserved ejection fraction in the ambulatory setting.

Multiple trials have not found medications to be an effective treatment, except for diuretics. If hypertension is present, it is treated according to evidence-based guidelines. Exercise and treatment by multidisciplinary teams may be helpful. Atrial fibrillation is treated using a rate-control strategy and appropriate anticoagulation. Revascularization is considered for patients with heart failure with preserved ejection fraction and coronary artery disease. The prognosis is comparable to that of heart failure with reduced ejection fraction and is worsened by higher levels of brain natriuretic peptide, older age, a history of myocardial infarction, and reduced diastolic function.

Additional therapies for HFpEF are needed.

### SUMMARY OF THE INVENTION

The present disclosure provides compositions and methods for treatment and prevention of cardiorenal damage. In particular, provided herein are compositions comprising proANP₃₁₋₆₇ for use in treating and preventing cardiorenal damage in subjects with heart failure.

Current medical treatments have not been successful in reducing morbidity and mortality in patients with HFpEF. We observed that proANP₃₁₋₆₇ peptide (VASTIRAS™) exerts beneficial cardiorenal actions in established HFpEF animals when we start treating them in their pre-HFpEF stage (suffering from cardiac hypertrophy, diastolic dysfunction, renal failure, metabolic syndrome and diabetes, including obesity, hypertension, hyperglycemia, glucosuria, and hyperlipidemia), and its effects are magnified when combined with ENTRESTO™. Specifically, we saw that chronic administration of proANP₃₁₋₆₇ peptide has unique protective effects on the kidney and heart structure and function. The combination proANP₃₁₋₆₇ with ENTRESTO™ showed rescued cardiac and renal phenotypes and functions, accompanied by lower animal body weight, and reduced pulmonary oedema, demonstrating a novel therapeutic strategy able to prevent and treat the harmful signs and symptoms of HFpEF even in a severe clinical model associated with overt metabolic disease.

For example, in some embodiments, provided herein is a method of treating or preventing one or more signs or symptoms of heart failure with preserved ejection fraction (HFpEF), comprising: administering a proANP₃₁₋₆₇ peptide of SEQ ID NO: 1 (e.g., VASTIRAS™) to a subject in need thereof.

For example, in some embodiments, provided herein is a method of treating or preventing one or more signs or symptoms of heart failure with preserved ejection fraction (HFpEF), comprising: administering a proANP₃₁₋₆₇ peptide to a subject with pre-HFpEF (e.g., a subject with metabolic syndrome).

The present disclosure is not limited to particular signs or symptoms of HFpEF. Examples include but are not limited to, end organ damage, cardiac adverse remodelling, metabolic syndrome, and impaired cardiac function. In some embodiments, the subject has one or more of hypertension, diabetes, renal failure, cardiac hypotrophy, diastolic dysfunction, metabolic syndrome, or obesity.

In some embodiments, the subject has pre HFpEF or overt HFpEF. In some embodiments, the administering prevents the development of HFpEF in the subject. In some embodiments, the administering improves cardiac and/or renal structure and function in the subject. In some embodiments, the administering results in a decrease in body weight and/or reduced pulmonary oedema. In some embodiments, the administering does not affect systemic blood pressure.

The present disclosure is not limited to a particular administration route or dosage of the proANP₃₁₋₆₇ peptide. Examples include but are not limited to, parenteral, subcutaneous, intravenous, oral, or intranasal delivery. Exemplary dosages are a dosage in the range of 25 to 2000 ng/kg/day.

In further embodiments, one or more additional agents useful, necessary, or sufficient in the treatment of HFpEF or related conditions are administered. For example, in some embodiments, an antihypertensive drug is administered. In some embodiments, ENTRESTO™ (i.e., a combination of sacubitril and valsartan) is administered.

Additional embodiments provide the use of a proANP₃₁₋₆₇ peptide in the manufacture of a medicament for treatment or prevention of HFpEF or a composition comprising a proANP₃₁₋₆₇ peptide for use in treatment or prevention of HFpEF.

Yet other embodiments provide the use of a composition comprising a proANP₃₁₋₆₇ peptide in combination with a composition comprising ENTRESTO™ (i.e., a combination of sacubitril and valsartan) for treatment or prevention of HFpEF or a composition comprising a proANP₃₁₋₆₇ peptide and a composition comprising ENTRESTO™ (i.e., a combination of sacubitril and valsartan) for use in treatment or prevention of HFpEF.

Additional embodiments are described herein.

### BRIEF DESCRIPTION OF THE DRAWINGS

FIG. 1 shows an overview of HFpEF syndrome.

FIG. 2 shows survival data for rats treated with proANP₃₁₋₆₇ and/or ENTRESTO™.

FIG. 3 shows autopsy data for rats treated with proANP₃₁₋₆₇ and/or ENTRESTO™.

FIG. 4 shows autopsy data for rats treated with proANP₃₁₋₆₇ and/or ENTRESTO™.

FIG. 5 shows blood pressure data for rats treated with proANP₃₁₋₆₇ and/or ENTRESTO™.

FIG. 6 shows invasive blood pressure data for rats treated with proANP₃₁₋₆₇ and/or ENTRESTO™.

FIG. 7 shows renal endpoint ultrasound data for rats treated with proANP₃₁₋₆₇ and/or ENTRESTO™.

FIG. 8 shows renal endpoint molecular biochemistry data for rats treated with proANP₃₁₋₆₇ and/or ENTRESTO™.

FIG. 9 shows renal endpoint molecular biochemistry data for rats treated with proANP₃₁₋₆₇ and/or ENTRESTO™.

FIG. 10 shows renal endpoint histology data for rats treated with proANP₃₁₋₆₇ and/or ENTRESTO™.

FIG. 11 shows renal structure and function histology data for rats treated with proANP₃₁₋₆₇ and/or ENTRESTO™.

FIG. 12 shows renal endpoint RT-PCR data for rats treated with proANP₃₁₋₆₇ and/or ENTRESTO™.

FIG. 13 shows renal endpoint ELISA data for rats treated with proANP₃₁₋₆₇ and/or ENTRESTO™.

FIG. 14 shows cardiac endpoint ultrasound data for rats treated with proANP₃₁₋₆₇ and/or ENTRESTO™.

FIG. 15 shows cardiac endpoint ultrasound data for rats treated with proANP₃₁₋₆₇ and/or ENTRESTO™.

FIG. 16 shows cardiac endpoint ultrasound data for rats treated with proANP₃₁₋₆₇ and/or ENTRESTO™.

FIG. 17 shows cardiac endpoint ultrasound data for rats treated with proANP₃₁₋₆₇ and/or ENTRESTO™.

FIG. 18 shows cardiac endpoint data for rats treated with proANP₃₁₋₆₇ and/or ENTRESTO™.

FIG. 19 shows cardiac endpoint histology data for rats treated with proANP₃₁₋₆₇ and/or ENTRESTO™.

FIG. 20 shows cardiac endpoint RT-PCR data for rats treated with proANP₃₁₋₆₇ and/or ENTRESTO™.

FIG. 21 shows cardiac endpoint RT-PCR data for rats treated with proANP₃₁₋₆₇ and/or ENTRESTO™.

FIG. 22 shows cardiac endpoint RT-PCR data for rats treated with proANP₃₁₋₆₇ and/or ENTRESTO™.

FIG. 23 shows cardiac endpoint RT-PCR data for rats treated with proANP₃₁₋₆₇ and/or ENTRESTO™.

FIG. 24 shows cardiac endpoint RT-PCR and ELISA data for rats treated with proANP₃₁₋₆₇ and/or ENTRESTO™.

FIG. 25 is a schematic drawing showing the experimental design for Example 1.

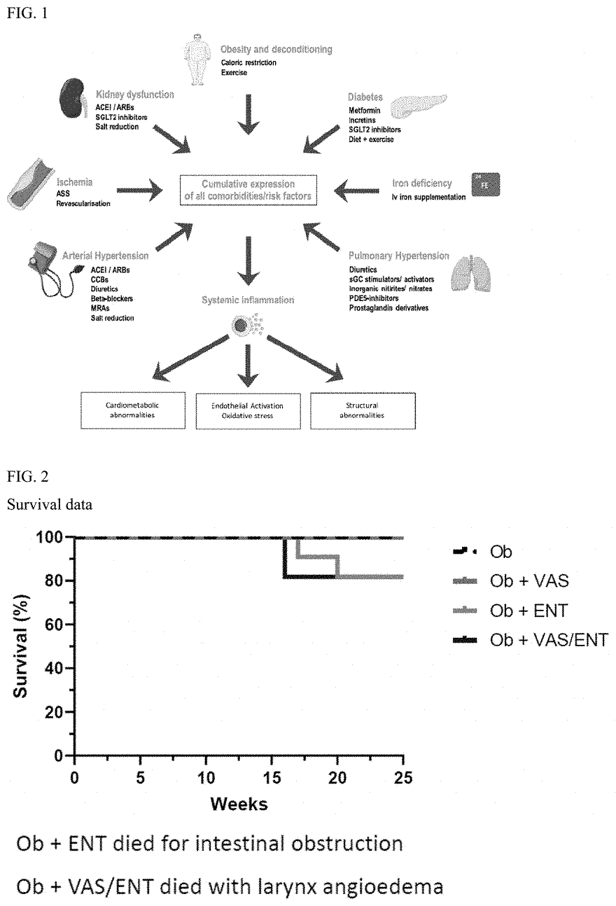

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., heart failure). Test compounds comprise both known and potential therapeutic compounds. A test compound can be determined to be therapeutic by screening using any suitable screening method.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a compound described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., those described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, or ex vivo.

As used herein, the term “toxic” refers to any detrimental or harmful effects on a cell or tissue as compared to the same cell or tissue prior to the administration of the toxicant.

“Amelioration” or “ameliorate” or “ameliorating” refers to a lessening of at least one indicator, sign, or symptom of an associated disease, disorder, or condition. The severity of indicators may be determined by subjective or objective measures, which are known to those skilled in the art.

The term “cardiac remodelling” refers to a group of molecular, cellular and interstitial changes that manifest as changes in the size, shape and function of the heart. These changes have a negative impact on cardiac function and eventually lead to heart failure.

### DETAILED DESCRIPTION OF THE INVENTION

The present disclosure provides compositions and methods for treatment and prevention of cardiorenal damage. In particular, provided herein are compositions comprising proANP₃₁₋₆₇ for use in treating and preventing cardiorenal damage in subjects with heart failure.

There are currently no drugs available on the market that can prevent adverse cardiac remodelling without lowering blood pressure. Indeed, most of the drugs indicated for anti-cardiac remodelling have hypotensive effects that can lead to adverse events in those patients with already unstable cardiovascular health and renal insufficiency.

Accordingly, provided herein is the use of proANP₃₁₋₆₇ (SEQ ID NO: 1; VASTIRAS™, Madeleine Pharmaceuticals, Madison, Wis.) to treat heart failure with preserved ejection fraction (HFpEF). VASTIRAS™ is the recombinant form of a human hormone, has demonstrated a unique safety profile when administered to patients. The VASTIRAS™ mechanism of action are independent of cGMP activation and differ from those of currently available medications, including other under-development designer peptides, like CDNP and MANP, which all reduce systemic blood pressure by activation of cGMP.

The present disclosure is not limited to particular signs or symptoms of HFpEF. Examples include but are limited to, end organ damage, cardiac adverse remodelling, and impaired cardiac function. In some embodiments, the subject has one or more of hypertension, diabetes, renal failure, cardiac hypotrophy, diastolic dysfunction, or obesity.

In some embodiments, the subject has pre HFpEF or overt HFpEF. In some embodiments, the administering prevents the development of HFpEF in the subject. For example, the subject to be treated according to the disclosure may be at risk of heart failure (but not display any overt symptoms of heart failure). In this embodiment, the subject is treated according to the disclosure to prevent the development of heart failure, or to reduce the risk of heart failure developing. In another embodiment, as detailed above, the subject treated has early-stage heart failure (with a severity level of NYHA class I or class II). In this embodiment, the subject is treated according to the disclosure to prevent the early-stage heart failure from progressing to moderate or severe heart failure (i.e., with a severity level of NYHA class III or class IV), or to reduce the risk of the heart failure progressing in this manner.

In some embodiments, the administering improves cardiac and/or renal structure and function in the subject. In some embodiments, the administering results in a decrease in body weight and/or reduced pulmonary oedema. In some embodiments, the administering does not affect systemic blood pressure.

The present disclosure is not limited to a particular administration route or dosage of the proANP₃₁₋₆₇ peptide. Examples include but are not limited to, parenteral, subcutaneous, intravenous, oral, or intranasal delivery. Exemplary dosages are a dosage in the range of 25 to 2000 ng/kg/day. Additional delivery systems and dosage regimens are provided below.

In further embodiments, one or more additional agents useful, necessary, or sufficient in the treatment of HFpEF or related conditions are administered. For example, in some embodiments, an antihypertensive drug (e.g., an ACE inhibitor, beta-blocker, calcium channel blocker or angiotensin receptor blocker (ARB)) is administered. In some embodiments, a diuretic (e.g., loop diuretics, thiazides, carbonic anhydrase inhibitors, potassium-sparing diuretics, calcium-sparing diuretics, osmotic diuretics and low ceiling diuretics) is administered. In some embodiments, ENTRESTO™, a combination of sacubitril and valsartan (Novartis, East Hanover, N.J.) is administered. In some embodiments, the combination therapy allows the dose of the additional agent to be decreased (e.g., to reduce side effects).

Natriuretic peptides (NPs) secreted by the heart reduce blood pressure, improve kidney function, and prevent adverse cardiac remodelling. However, in hypertension their protective actions are diminished (Belluardo et al., American Journal of Physiology—Heart and Circulatory Physiology 291(4): H1529-1535, 2006) due to reduced gene expression (Ferrari et al., Journal of Clinical Endocrinology and Metabolism 71(4): 944-951, 1990), as well as accumulation of altered molecular forms with reduced biological value (Macheret et al., Journal of the American College of Cardiology 60(16): 1558-1565, 2012). When used as therapeutic agents, the ring structured natriuretic peptides, including atrial-natriuretic-peptide (ANP)₁₋₂₈ and B-type-natriuretic-peptide (BNP)₁₋₃₂, exhibit intense hypotensive effects.

ANP is synthesised as a preprohormone, known as preproANP, a 151 amino acid polypeptide. Cleavage of the N-terminal signal sequence yields proANP, a 126 amino acid polypeptide. ProANP is stored in atrial granules. Following release from these granules, proANP is cleaved by the serine protease corin to yield the mature ANP peptide α-ANP, which constitutes the C-terminal 28 amino acids of proANP. The resulting N-terminal fragment of this cleavage reaction (proANP₁₋₉₈) is subsequently cleaved into three fragments: proANP₁₋₃₀, proANP₃₁₋₆₇ (SEQ ID NO: 1) and proANP₇₉₋₉₈ (Potter et al., Handbook of Experimental Pharmacology 191: 341-366, 2009; De Palo et al., Clinical Chemistry 46(6): 843-847, 2000).

Mature α-ANP is produced by the heart when under stress and acts to decrease blood pressure by vasodilation and by increasing natriuresis and diuresis. These activities are primarily mediated by its binding to natriuretic peptide receptor-A (NPR-A). All mature natriuretic peptides (including α-ANP) have a 17 amino acid disulphide ring, which is required for their binding to NPR-A (Potter et al., supra).

ProANP₃₁₋₆₇ is a linear peptide which has a unique mode of action. ProANP₃₁₋₆₇ does not contain the 17 amino acid ring found in mature natriuretic peptides and does not exert its functions by activating the classic natriuretic peptide receptors (NPR-A and NPR-B). It appears to be resistant to degradation by neprilysin and other endopeptidases and is excreted largely intact in urine. ProANP₃₁₋₆₇ is known to stimulate prostaglandin E₂ (PGE₂) formation in the kidney (Chiou & Vesely, Endocrinology 136(5): 2033-2039, 1995). Renal PGE₂ acts to increase renal blood flow and glomerular filtration rate. Recombinant proANP₃₁₋₆₇ has been shown to protect against ischemia-induced acute tubular necrosis and renal failure (Clark et al., American Journal of Physiology—Heart and Circulatory Physiology 278: H1555-1564, 2000). On this basis, proANP₃₁₋₆₇ (also known as vessel dilator, VSDL) has been found to be a safe and effective treatment for renal impairment in patients with congestive heart failure (Delacroix et al., Journal of the American College of Cardiology 67(13): Abstract 1351, 2016).

The subject treated according to the present disclosure may be any animal with a heart. The subject may be any mammal. It may be any domestic, livestock or sports animal. Preferably however, the subject is a human.

The subject treated according to the present disclosure may have any other condition that puts the subject at risk of developing heart failure. For instance, the subject may have a heart valve disorder. Heart valve disorders frequently drive cardiac remodelling, by putting increased pressure on the relevant chamber of the heart. Heart valve disorders include aortic stenosis, in which a narrowing of the aortic valve opening restricts passage of blood from the left ventricle to the aorta, causing increased pressure on the left ventricle and thus left ventricular hypertrophy. Another heart valve disorder is aortic insufficiency (also known as aortic regurgitation), in which the aortic valve leaks, allowing blood to pass through in the reverse direction (i.e., from the aorta into the left ventricle). This leakage causes increased pressure on the left ventricle, and thus left ventricular hypertrophy. Similarly, disorders of the mitral valve (particularly mitral stenosis and mitral regurgitation) can cause left atrial enlargement, as they cause increased pressure on the left atrium; disorders of the tricuspid valve (particularly tricuspid valve stenosis and tricuspid valve regurgitation) can cause right atrial enlargement; and disorders of the pulmonary valve (particularly pulmonary valve stenosis and pulmonary valve regurgitation) can cause right ventricular hypertrophy.

The subject treated according to the present disclosure may have an endocrine (hormonal) disorder. Several endocrine disorders are known to be associated with cardiac remodelling, as described below. An endocrine disorder is any disorder of the endocrine system, in particular a disorder associated with hypersecretion or hyposecretion of one or more hormones from an endocrine gland. More generally the subject may have a metabolic disorder. This may include metabolic syndrome, or insulin resistance or any condition associated with insulin resistance.

One such endocrine disorder is diabetes mellitus (diabetes). As is known to the skilled person, there are two types of diabetes: type 1 (which is an autoimmune condition in which an autoimmune response causes destruction of the insulin-producing beta cells of the pancreas); and type 2 (which is caused by insulin resistance, in which cells fail to respond properly to insulin). Both types of diabetes, but particularly type 2, are associated with an increased risk of developing heart failure, indicating that diabetes promotes cardiac remodelling. The subject treated according to the disclosure may thus be a subject with diabetes. In particular, the subject treated according to the disclosure may have type 2 diabetes.

Obesity is a significant risk factor for both hypertension and diabetes, meaning obese individuals are at particular risk of heart failure. The present disclosure may thus be used to treat or prevent cardiac remodelling in an obese subject.

Another endocrine disorder is thyroid disease. Both hypothyroidism and hyperthyroidism are associated with cardiac remodelling and heart failure. Thus, the subject may have hypothyroidism or hyperthyroidism. Parathyroid disease is also associated with cardiac remodelling: hyperparathyroidism is associated with hypertension and obesity; hypoparathyroidism is associated with decreased cardiac performance, and increased risk of developing dilated cardiomyopathy. Thus, the subject treated according to the method disclosed herein may have parathyroid disease, e.g., hyperparathyroidism or hypoparathyroidism.

In a particular embodiment the subject treated according to the present disclosure is a subject who has suffered a myocardial infarction. In another embodiment, the subject treated according to the disclosure has myocarditis (inflammation of the heart muscle, generally caused by a viral infection). In another embodiment the subject treated according to the disclosure has cardiomyopathy, which is described above. Cardiomyopathy may be caused by e.g., a viral infection, hypertension, a disease of the tissue, etc. Cardiomyopathy may alternatively be a genetic condition (familial cardiomyopathy). Subjects with either familial cardiomyopathy or non-familial cardiomyopathy may be treated according to the present disclosure.

In another embodiment the subject treated according to the disclosure has an arrhythmia. Arrhythmias are conditions in which the heartbeat is not properly coordinated (i.e., does not have a correct rhythm). Arrhythmia may cause a slow heartbeat (bradycardia), a fast heartbeat (tachycardia) or an irregular heartbeat (fibrillation). Arrhythmias may be caused by an array of conditions.

In another embodiment, the subject treated according to the present disclosure has chronic obstructive pulmonary disease (COPD). COPD is a condition which causes breathing difficulties, primarily caused by damage to the airways as a result of smoking. A common complication of COPD is pulmonary hypertension (specifically of WHO Group III, pulmonary hypertension linked with lung disease or hypoxia), which as described above causes right ventricular hypertrophy.

In another embodiment the subject may be a drug or alcohol user. In particular, the subject may be abusing alcohol or drugs, or excessively using alcohol or drugs. This may be over a prolonged or extended period of time, particular in the case of alcohol, e.g., over a period of years. The drugs may be recreational drugs (e.g., cocaine or amphetamines) or anabolic steroids.

In one particular embodiment, the present disclosure provides a method of prophylactic or therapeutic treatment of end organ damage in heart failure patients with preserved ejection fraction comprising the step of administering a pharmaceutical dose of proANP₃₁₋₆₇ to a patient suffering from metabolic syndrome.

In another embodiment the subject may be undergoing cardiotoxic therapy, that is to say medical treatment which is damaging to the heart (i.e., which may cause cardiac remodelling). For instance, many chemotherapy agents commonly used in cancer treatment are cardiotoxic, including anthracyclines (such as doxorubicin), alkylating agents (such as cisplatin, carboplatin and cyclophosphamide) topoisomerase inhibitors, antimetabolites (such as 5-fluorouracil) and monoclonal antibodies. Thus, in a particular embodiment the subject is undergoing chemotherapy for treatment of cancer. Radiotherapy, also commonly used in cancer treatment, is also cardiotoxic. Thus, in another particular embodiment the subject is undergoing radiotherapy for treatment of cancer. By “undergoing” chemotherapy or radiotherapy is meant a subject who is currently receiving a course of chemotherapy or radiotherapy, who is to begin a course of chemotherapy or radiotherapy (e.g., has been prescribed chemotherapy or radiotherapy), or who has recently completed a course of chemotherapy or radiotherapy. Thus, in this context, the polypeptide used according to the present disclosure may be administered to the subject in advance of their chemo/radiotherapy course, during their chemo/radiotherapy course and/or after the completion of their chemo/radiotherapy course.

Typically, the proANP₃₁₋₆₇ compound for use according to the disclosure will be administered as a composition consisting of a solution or suspension of the compound in a pharmaceutically acceptable carrier, diluent or excipient. However, it will be readily appreciated by the person skilled in the art that the compound may be bound to or associated with a carrier molecule (e.g., a carrier protein or fusion partner such as human serum albumin (HSA), a polysaccharide (e.g., dextran) or a polyether (e.g., polyethylene glycol)) in order to modulate the biological activity and/or serum half-life of the compound.

Suitable pharmaceutically acceptable diluents, carriers and excipients are well known in the art. For instance, suitable excipients include lactose, maize starch or derivatives thereof, stearic acid or salts thereof, vegetable oils, waxes, fats and polyols. Suitable carriers or diluents include carboxymethylcellulose (CMC), methylcellulose, hydroxypropylmethylcellulose (HPMC), dextrose, trehalose, polyvinyl alcohol, pharmaceutical grade starch, mannitol, lactose, magnesium stearate, sodium saccharin, talcum, cellulose, glucose, sucrose (and other sugars), magnesium carbonate, gelatine, oil, alcohol, detergents and emulsifiers such as polysorbates. Stabilising agents, wetting agents, sweeteners etc. may also be used.

Liquid pharmaceutical compositions, whether they be solutions, suspensions or other like form, may include one or more of the following: sterile diluents such as water, saline solution (preferably physiological, i.e. isotonic), Ringer's solution, fixed oils such as synthetic mono- or diglycerides which may serve as a solvent or suspending medium, polyethylene glycols, glycerine, propylene glycol or other solvents; antibacterial agents such as benzyl alcohol or methyl paraben; antioxidants such as ascorbic acid or sodium bisulfate; chelating agents such as EDTA; buffers such as acetates, citrates or phosphates and agents for the adjustment of tonicity such as sodium chloride or dextrose. A parenteral preparation can be enclosed in ampoules, disposable syringes or multiple dose vials made of glass or plastic. A pharmaceutical composition comprising a proANP₃₁₋₆₇ compound for use according to the disclosure is preferably sterile.

The administration of the proANP₃₁₋₆₇ compound according to the disclosure may be performed by any suitable route. For instance, the proANP₃₁₋₆₇ compound may be administered to the subject intravenously (IV), subcutaneously (sc) or intranasally. IV administration is particularly suitable in the hospital setting, and sc administration and intranasal administration are suitable both within the hospital setting and in the community. The proANP₃₁₋₆₇ compound is preferably administered as a bolus and/or sustained infusion (e.g., for a period of 30 minutes, 1 hour or longer). For example, infusion may be via a standard catheter or implantable drug port (e.g. a Port-a-Cath®; Smiths Medical MD, Inc., USA), or otherwise achieved using a drug infusion pump (e.g. an implantable drug infusion pump such as an Alzet® osmotic pump (Durect Corporation, USA) or a Duros® device (Intarcia Therapeutics, Inc., USA), or a drug infusion pump for subcutaneous (sc) administration such as a Paradigm™ device (Medtronic, USA), all of which can provide a controlled release of the proANP₃₁₋₆₇ compound. The use of an implantable drug port or drug infusion pump is particularly well suited for long term or extended treatments. Typically, the proANP₃₁₋₆₇ compound will be infused at a constant rate. However, in some cases it may be desirable to employ a drug infusion pump employing a feedback control mechanism (e.g., a feedback linked to measurement of oedema (in the lung) or other surrogate marker) to control release of the proANP₃₁₋₆₇ compound.

The dosage of the proANP₃₁₋₆₇ compound administered according to the disclosure may be determined by factors such as the medical condition of the patient (e.g., what medical conditions the patient suffers from). Appropriate dosages may be determined as a factor of the size of the patient. The skilled clinician will be able to calculate an appropriate dose for a patient based on all relevant factors, e.g., age, height, weight, the condition to be treated and its severity.

Accordingly, in an embodiment of the disclosure the proANP₃₁₋₆₇ compound is administered subcutaneously to the subject at a dosage in the range 25 to 2000 ng/kg/day. In other embodiments the maximum subcutaneous dosage may be 1900, 1800, 1700, 1600, 1500, 1400, 1300, 1200, 1100, 1000, 900, 800, 700, 600, 500, 400, 300, 200, 100, 95, 90, 85, 80, 75, 70, 65, 60, 55 or 50 ng/kg/day. In other embodiments the minimum subcutaneous dosage may be 30, 35, 40, 45, 50, 55, 60, 65, 70, 75, 80, 85, 90, 95, 100, 200, 300, 400, 500, 600, 700, 800, 900, 1000, 1100, 1200, 1300, 1400 or 1500 ng/kg/day. In some embodiments of the disclosure, the proANP₃₁₋₆₇ compound is administered subcutaneously to the subject at a dosage in the range 25 to 50 ng/kg/day, 25 to 75 ng/kg/day, 25 to 80 ng/kg/day, 25 to 85 ng/kg/day, 25 to 90 ng/kg/day, 25 to 95 ng/kg/day, 25 to 100 ng/kg/day, 25 to 200 ng/kg/day, 25 to 500 ng/kg/day, 25 to 1000 ng/kg/day or 25 to 1500 ng/kg/day.

In other embodiments of the disclosure, the proANP₃₁₋₆₇ compound is administered to the subject subcutaneously at a dosage in the range 50 to 75 ng/kg/day, 50 to 80 ng/kg/day, 50 to 85 ng/kg/day, 50 to 90 ng/kg/day, 50 to 95 ng/kg/day, 50 to 100 ng/kg/day, 50 to 200 ng/kg/day, 50 to 500 ng/kg/day, 50 to 1000 ng/kg/day or 50 to 1500 ng/kg/day. In other embodiments of the disclosure, the proANP₃₁₋₆₇ compound is administered to the subject subcutaneously at a dosage in the range 100 to 200 ng/kg/day, 100 to 300 ng/kg/day, 100 to 400 ng/kg/day, 100 to 500 ng/kg/day, 100 to 1000 ng/kg/day, 100 to 1500 ng/kg/day or 100 to 2000 ng/kg/day. In other embodiments of the disclosure, the proANP₃₁₋₆₇ compound is administered to the subject subcutaneously at a dosage in the range 250 to 500 ng/kg/day, 250 to 750 ng/kg/day, 250 to 1000 ng/kg/day, 250 to 1500 ng/kg/day or 250 to 2000 ng/kg/day. In other embodiments of the disclosure, the proANP₃₁₋₆₇ compound is administered to the subject subcutaneously at a dosage in the range 500 to 1000 ng/kg/day, 500 to 1500 ng/kg/day or 500 to 2000 ng/kg/day. In other embodiments of the disclosure, the proANP₃₁₋₆₇ compound is administered to the subject subcutaneously at a dosage in the range 1000 to 1500 ng/kg/day or 1000 to 2000 ng/kg/day.

In preferred embodiments of the disclosure, the proANP₃₁₋₆₇ compound is administered to the subject subcutaneously at a dosage less than 100 ng/kg/day, e.g., at a dosage in the range 25 to 50 ng/kg/day, 25 to 75 ng/kg/day, 25 to 80 ng/kg/day, 25 to 85 ng/kg/day, 25 to 90 ng/kg/day or 25 to 95 ng/kg/day.

The target plasma concentration of the proANP₃₁₋₆₇ compound may alternatively be achieved by intravenous administration of 5 to 1000 ng/kg/day of the proANP₃₁₋₆₇ compound. Accordingly, in an embodiment of the disclosure the proANP₃₁₋₆₇ compound is administered intravenously to the subject at a dosage in the range 5 to 1000 ng/kg/day. In other embodiments the maximum intravenous dosage may be 900, 800, 700, 600, 500, 400, 300, 200, 100, 95, 90, 85, 80, 75, 70, 65, 60, 55, 50, 45, 40, 35, 30, 25 or 20 ng/kg/day. In other embodiments the minimum intravenous dosage may be 10, 15, 20, 25, 30, 35, 40, 45, 50, 55, 60, 65, 70, 75, 80, 85, 90, 95, 100, 200, 300, 400 or 500 ng/kg/day.

In some embodiments of the disclosure, the proANP₃₁₋₆₇ compound is administered to the subject intravenously at a dosage in the range 5 to 15 ng/kg/day, 6 to 18 ng/kg/day, 6 to 20 ng/kg/day, 6 to 23 ng/kg/day, 6 to 25 ng/kg/day, 6 to 30 ng/kg/day, 6 to 40 ng/kg/day, 6 to 50 ng/kg/day, 6 to 75 ng/kg/day, 6 to 100 ng/kg/day, 6 to 250 ng/kg/day or 6 to 500 ng/kg/day. In other embodiments the proANP₃₁₋₆₇ compound is administered to the subject intravenously at a dosage in the range 10 to 18 ng/kg/day, 10 to 20 ng/kg/day, 10 to 25 ng/kg/day, 10 to 30 ng/kg/day, 10 to 40 ng/kg/day, 10 to 50 ng/kg/day, 10 to 75 ng/kg/day, 10 to 100 ng/kg/day, 10 to 250 ng/kg/day or 10 to 500 ng/kg/day. In other embodiments the proANP₃₁₋₆₇ compound is administered to the subject intravenously at a dosage in the range 25 to 50 ng/kg/day, 25 to 75 ng/kg/day, 25 to 100 ng/kg/day, 25 to 250 ng/kg/day or 25 to 500 ng/kg/day.

In preferred embodiments of the disclosure, the proANP₃₁₋₆₇ compound is administered to the subject intravenously at a dosage up to 25 ng/kg/day, e.g. at a dosage in the range 5 to 10 ng/kg/day, 5 to 15 ng/kg/day, 5 to 20 ng/kg/day, 5 to 25 ng/kg/day, 6 to 10 ng/kg/day, 6 to 15 ng/kg/day, 6 to 18 ng/kg/day, 6 to 20 ng/kg/day, 6 to 23 ng/kg/day, 6 to 25 ng/kg/day, 10 to 15 ng/kg/day, 10 to 20 ng/kg/day or 10 to 25 ng/kg/day.

The target plasma concentration of the proANP₃₁₋₆₇ compound may alternatively be achieved by intranasal administration of 5 to 1000 ng/kg/day of the proANP₃₁₋₆₇ compound. Accordingly, in an embodiment of the disclosure the proANP₃₁₋₆₇ compound is administered intranasally to the subject at a dosage in the range 5 to 1000 ng/kg/day. Accordingly, in an embodiment of the disclosure the proANP₃₁₋₆₇ compound is administered intranasally to the subject at a dosage in the range 5 to 1000 ng/kg/day. In other embodiments the maximum intranasal dosage may be 900, 800, 700, 600, 500, 400, 300, 200, 100, 95, 90, 85, 80, 75, 70, 65, 60, 55, 50, 45, 40, 35, 30, 25 or 20 ng/kg/day. In other embodiments the minimum intranasal dosage may be 10, 15, 20, 25, 30, 35, 40, 45, 50, 55, 60, 65, 70, 75, 80, 85, 90, 95, 100, 200, 300, 400 or 500 ng/kg/day.

In some embodiments of the disclosure, the proANP₃₁₋₆₇ compound is administered to the subject intranasally at a dosage in the range 5 to 15 ng/kg/day, 5 to 20 ng/kg/day, 5 to 25 ng/kg/day, 5 to 30 ng/kg/day, 5 to 40 ng/kg/day, 5 to 50 ng/kg/day, 5 to 75 ng/kg/day, 5 to 100 ng/kg/day, 5 to 250 ng/kg/day or 5 to 500 ng/kg/day. In other embodiments the proANP₃₁₋₆₇ compound is administered to the subject intranasally at a dosage in the range 8 to 15 ng/kg/day, 8 to 20 ng/kg/day, 8 to 25 ng/kg/day, 8 to 30 ng/kg/day, 8 to 40 ng/kg/day, 8 to 50 ng/kg/day, 8 to 75 ng/kg/day, 8 to 100 ng/kg/day, 8 to 250 ng/kg/day or 8 to 500 ng/kg/day. In other embodiments the proANP₃₁₋₆₇ compound is administered to the subject intranasally at a dosage in the range 10 to 20 ng/kg/day, 10 to 25 ng/kg/day, 10 to 30 ng/kg/day, 10 to 40 ng/kg/day, 10 to 50 ng/kg/day, 10 to 75 ng/kg/day, 10 to 100 ng/kg/day, 10 to 250 ng/kg/day or 10 to 500 ng/kg/day. In other embodiments the proANP₃₁₋₆₇ compound is administered to the subject intranasally at a dosage in the range 25 to 50 ng/kg/day, 25 to 75 ng/kg/day, 25 to 100 ng/kg/day, 25 to 250 ng/kg/day or 25 to 500 ng/kg/day.

In preferred embodiments of the disclosure, the proANP₃₁₋₆₇ compound is administered to the subject intranasally at a dosage up to 30 ng/kg/day, e.g. at a dosage in the range 5 to 10 ng/kg/day, 5 to 15 ng/kg/day, 5 to 20 ng/kg/day, 5 to 25 ng/kg/day, 5 to 30 ng/kg/day, 8 to 15 ng/kg/day, 8 to 20 ng/kg/day, 8 to 25 ng/kg/day, 8 to 30 ng/kg/day, 10 to 15 ng/kg/day, 10 to 20 ng/kg/day, 10 to 25 ng/kg/day or 10 to 30 ng/kg/day.

The disclosure may be further understood by reference to the non-limiting examples below, and the figures.

### EXPERIMENTAL

### Example 1

### Methods:

A hybrid rat that was a cross between a ZDF female and an SHHF male was utilized. The rat exhibited hypertension, type 2 diabetes, hyperlipidemia, nephropathy, and metabolic syndrome.

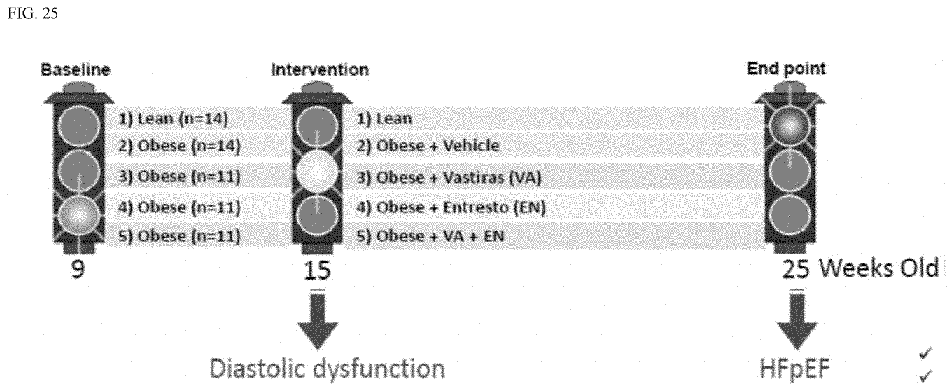

The study design is provided in FIG. 25. Briefly, we performed an interventional study in pre-HFpEF rats (15 weeks old) affected by obesity, diabetes, hypertension, diastolic dysfunction, and administered either vehicle, VASTIRAS™ (proANP₃₁₋₆₇; VAS; 50 ng/Kg/day administered subcutaneously via Alzet osmotic pump), ENTRESTO™ (ENT; 68 mg/Kg/day administered orally via gavage), or VAS+ENT (VAS/ENT).

SEQ ID NO: 1
(VAS; EVVPPQVLSEPNEEAGAALSPLPEVPPWTGEVSPAQR).

### Results:

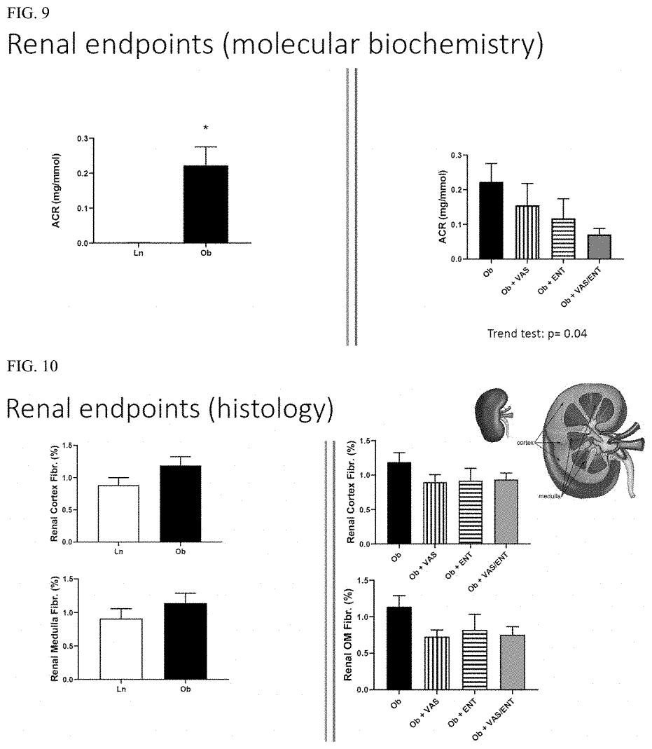

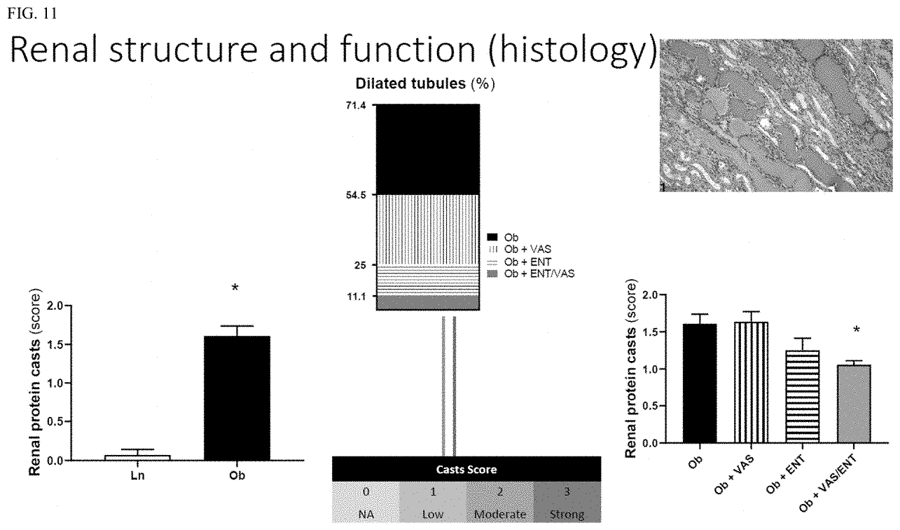

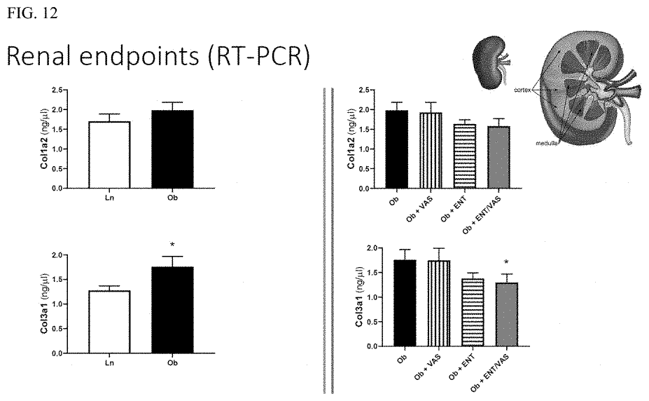

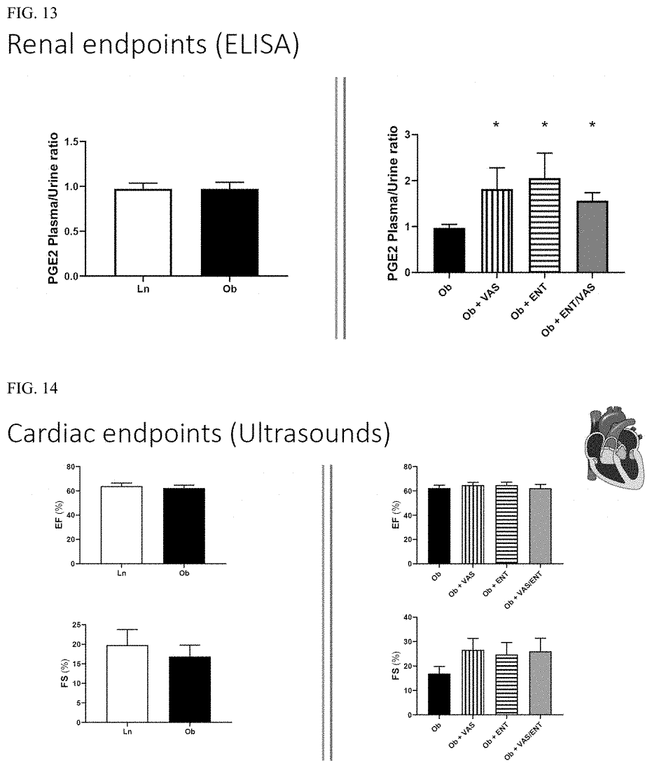

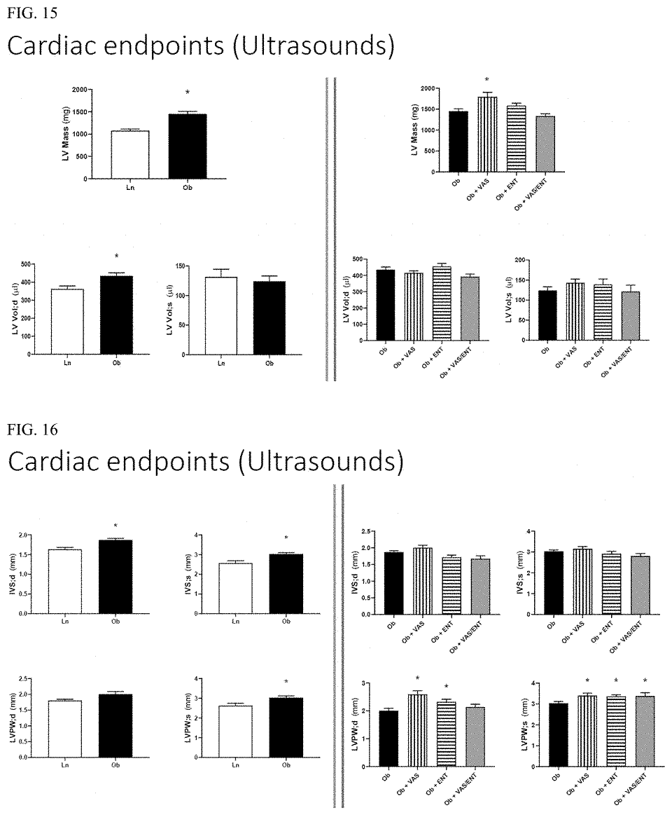

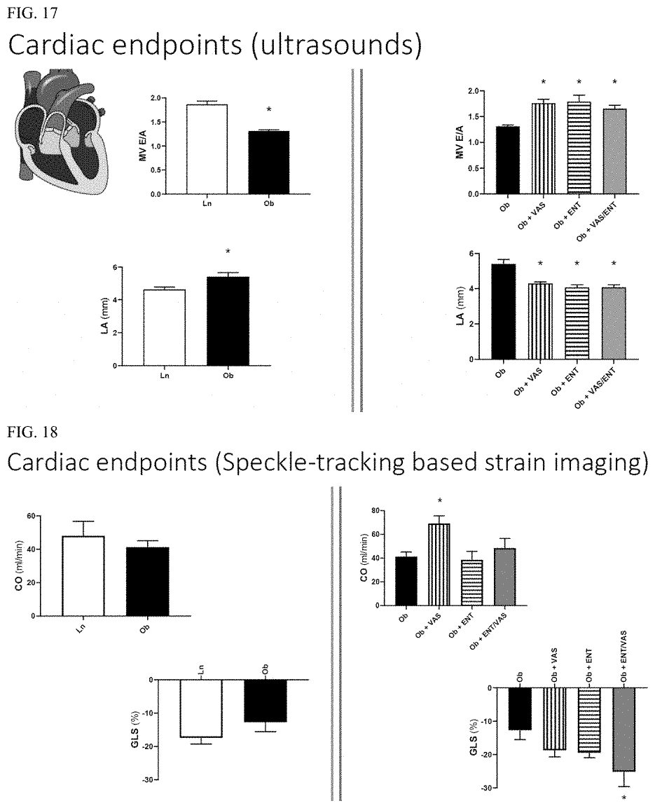

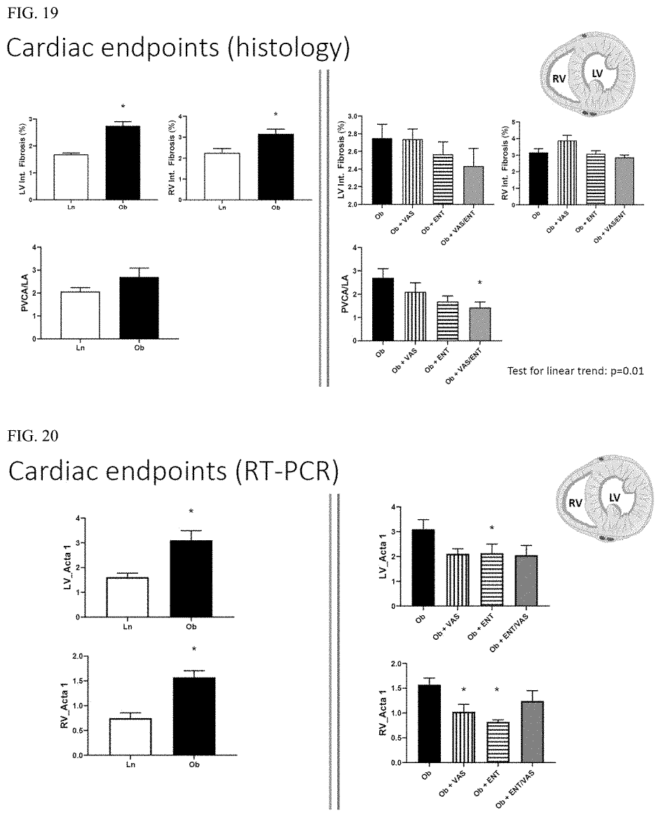

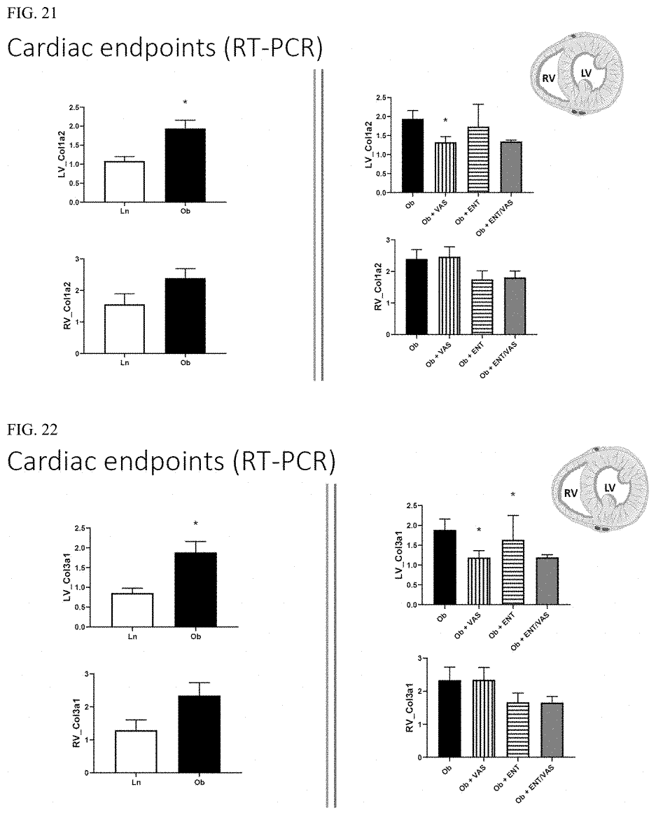

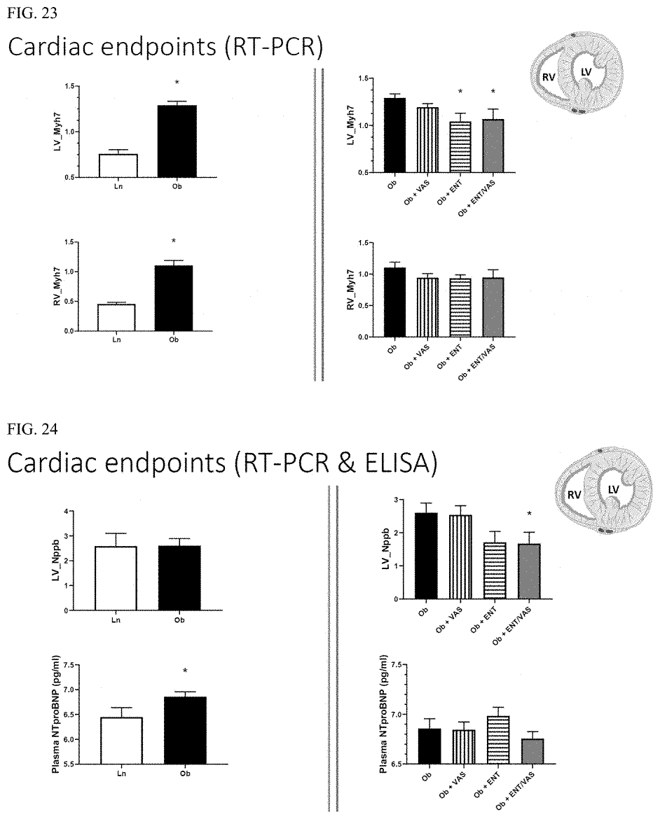

Results are shown in FIGS. 2-24.

At 25 weeks-old, ZSF1-Ob rats exhibited signs of HFpEF, including obesity, hypertension, renal failure and fibrosis, cardiac hypertrophy and diastolic dysfunction accompanied by fibrosis.

Elevated blood pressure was not affected by the treatment with VAS, but was reduced by ENT (FIGS. 5-6).

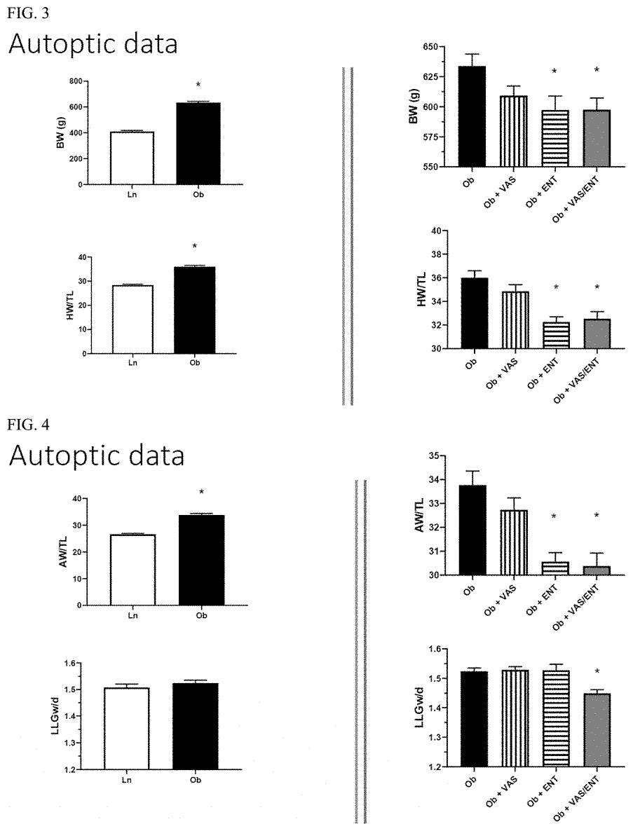

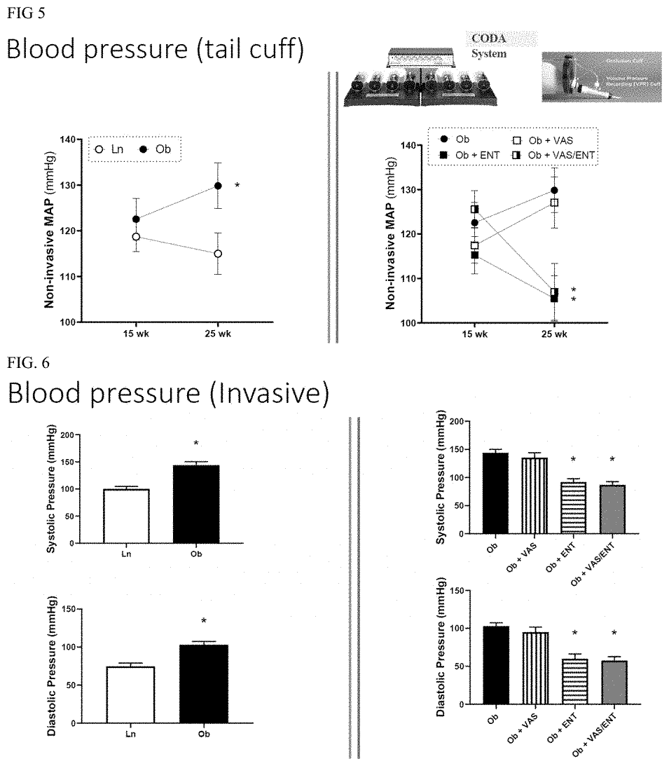

Rats treated with ENT exhibited lower body weight (BW), heart weight (HW)/tibia length (TL), atria weight (AW)/TL, and kidney weight (KW)/TL FIGS. 3-4). VASTIRAS™ tended to decrease BW, HW/TL, AW/TL, and KW/TL. However, pulmonary edema was only reduced in the ZSF1-Ob+VAS/ENT group, as indexed by the ratio of wet to dry lung weight.

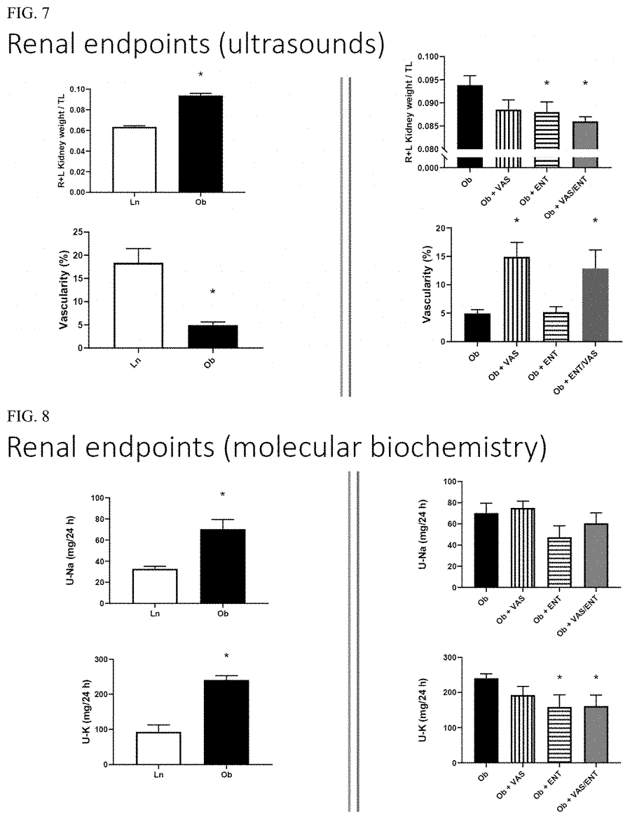

The ZSF1-Ob group was associated with decreased kidney vascularity, and increased Na⁺, K⁺ excretion, albumin/creatinine (ACR) ratio, dilated tubules, protein casts score, and expression of collagen 3a1 (Col3a1) (FIGS. 7-13). ENTRESTO™ decreased K⁺ excretion, ACR ratio, and the percent of dilated tubules, whereas VAS preserved renal vascularity. When combined, VAS and ENT preserved renal vascularity, reduced K⁺ excretion, decreased the ACR ratio, drastically lowered the percent of dilated tubules and renal protein casts score, and reduced the expression of Col3a1. Increased production of renal PGE₂ as shown by the ratio of plasma over urine concentration was observed in all treatment groups.

Cardiac ultrasounds of ZSF1-Ob rats exhibited no differences in the percent of left ventricle (LV) ejection fraction and fraction of shortening compared to lean animals (FIGS. 14-24). LV mass, LV thickness, and left atrium diameter were increased, along with increased diastolic dysfunction as indexed by mitral valve flow E/A ratio. All three treatment groups exhibited a rescued E/A ratio and atrium diameter similar to controls. VAS improved cardiac output and tended to increase global longitudinal strain (GLS). When combined with ENT, VAS further increased the GLS. Moderate cardiac fibrosis was detected in the ZSF1-Ob group. Perivascular collagen deposition was reduced by the combination of VAS and ENT. Expression of the heart failure biomarker gene nppb in the left ventricle was reduced by co-treatment of VAS and ENT.

In conclusion, our findings demonstrate that VAS has unique protective effects on kidney and heart structure and function that further enhance those of ENT, in a preclinical model of HFpEF with cardiorenal metabolic syndrome.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A method of treating or preventing one or more signs or symptoms of heart failure with preserved ejection fraction (HFpEF), comprising:
administering a proANP₃₁₋₆₇ peptide to a subject in need thereof.

2. The method of claim 1, wherein the one or more signs or symptoms are selected from the group consisting of end organ damage, cardiac adverse remodelling, and impaired cardiac function.

3. The method of claim 1, wherein the subject has one or more of hypertension, diabetes, renal failure, cardiac hypotrophy, diastolic dysfunction, metabolic syndrome, or obesity.

4. The method of claim 1, wherein the subject has pre HFpEF.

5. The method of claim 4, wherein said administering prevents the development of HFpEF in the subject.

6. The method of claim 1, wherein subject has HFpEF.

7. The method of claim 1, wherein the administering improves cardiac and/or renal structure and function.

8. The method of claim 1, wherein the administering results in a decrease in body weight and/or reduced pulmonary oedema.

9. The method of claim 1, wherein the proANP₃₁₋₆₇ peptide consists of the amino acid sequence set forth in SEQ ID NO: 1.

10. The method of claim 1, wherein the proANP₃₁₋₆₇ peptide is administered subcutaneously, intravenously, orally, or intranasally to the subject at a dosage in the range of 25 to 2000 ng/kg/day.

11. The method of claim 1, wherein the administering does not affect systemic blood pressure.

12. The method of claim 1, wherein the method further comprises administering a combination of sacubitril and valsartan to said subject.

13. The method of claim 1, wherein the method further comprises administering an antihypertensive drug to said subject.

14. A composition comprising a proANP₃₁₋₆₇ peptide, sacubitril and valsartan for use in treatment or prevention of HFpEF.
